(19) United States
(12) Patent Application Publication
(10) Pub. No.: US 2005/0266048 A1
(43) Pub. Date: Dec. 1, 2005

(54) Hemorrhoid treatment device

(76) Inventor: Jiro Takashima, Houston, TX (US)

Correspondence Address: John S. Egbert;Harrison & Egbert, 7th Floor, 412 Main Street, Houston, TX, 77002, US

(21) Appl. No.: 11/130,827

(22) Filed: May 17, 2005

Related U.S. Application Data
(60) Provisional application No. 60/571,820, filed on May 17, 2004.

(51) Int. Cl. A61B 17/32

(52) U.S. Cl. 424/436

(57) ABSTRACT

The hemorrhoid treatment device has a head and a shaft. The head has a spherical shape which is between 6 and 14 millimeters in diameter. The shaft has a cylindrical shape extending from the head. The diameter of the shaft is less than one half of the diameter of the head. The body has a length of between 25 and 45 millimeters.

### RELATED U.S. APPLICATIONS

The present application claims priority from U.S. Provisional Patent Application Ser. No. 60/571,820, filed on May 17, 2004, and entitled “HEMORRHOID TREATMENT DEVICE.”

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates to apparatus for the treatment of hemorrhoids. More particularly, the present invention relates to devices for massaging internal and external hemorrhoids by the self movement of the device. The present invention also relates to devices that are held within the anal canal and are driven by the peristaltic activity of the surface of the anal canal for the purpose of massaging internal and external hemorrhoids.

### BACKGROUND OF THE INVENTION

There are many anal treatment dilators and various mechanical devices, which are intended to facilitate the treatment of hemorrhoidal tissues. U.S. Pat. No. 4,263,914 massages the anal canal manually. U.S. Pat. No. 4,583,542 issued on Apr. 22, 1986 to Z. R. Boyd, describes a stationary device with a steep circumferential shoulder of the head and an outward tapered surface toward the large flange associated with the device. When the anal sphincter contracts, the lateral pressure applied on the outward tapered surface and the flange, will thrust the device out from the anus. This device is intended to stay in a stationary position when the anal sphincter relaxes. U.S. Pat. No. 5,797,950 to the present inventor utilizes anal sphincter contractions to massage the prostate by reciprocal linear movement of the apparatus held by a rod set against the perineum region. U.S. Pat. No. 6,589,193 to the present inventor utilizes the entire anal canal's static and dynamic lateral pressure and the peristaltic activity of the anal canal to massage the internal and external hemorrhoids. With the exception of U.S. Pat. No. 6,589,193, no previous invention mobilizes the apparatus to massage the internal and external hemorrhoids mostly by the activity of the pecten within the anal canal.

U.S. Design Pat. No. 428,488, issued on Jul. 18, 2000 shows a first ball and a second ball connected by a shaft. The shaft has a diameter that is proportionally large in comparison with the length of the shaft and the diameter of the heads. The diameter of the shaft is about one-third of the length of the shaft and about 60% of the diameter of one of the heads. The shaft has a central concave narrow section. The relatively large diameter of the shaft in combination with narrow section will be gripped by the pecten and will, accordingly, hamper free movement of the head throughout the internal hemorrhoidal area. The proportionally large diameter of the shaft in combination with the diameter of the head will contribute to undesired easy evacuation of the device from the anal canal.

It is an object of the present invention to facilitate the effects of combination of the rectal pressure and the peristaltic movement of the pecten inside the anal canal to gently massage hemorrhoids. The rectal pressure and the peristaltic activity within the pecten drive the device slightly back and forth. The pecten is a narrow, concaved portion of the anal canal. These slight movements effectively massage the internal and the external hemorrhoids without aggravating the condition. In addition, the peristaltic movement within the pecten consumes a large amount of energy, increasing fresh blood circulation to the anorectal area to improve hemorrhoidal conditions.

It is another object of the present invention to provide a short and small diameter device with a smooth surface mostly working only within the anal canal so as not to aggravate the condition of the hemorrhoids.

It is a further object of the present invention to provide a hemorrhoidal treatment device, which is easy to use, inexpensive and easy to manufacture.

### BRIEF SUMMARY OF THE INVENTION

A hemorrhoid treatment device comprises a head having a ball-like shape, and a shaft connected to the head. The shaft has a generally cylindrical surface extending from the head. The cylindrical surface should have a generally constant diameter extending between the heads. The diameter of the wide portion of the head is from 6 mm to 14 mm inclusive, and the length of the top of the head and the bottom of the head is from 6 mm to 14 mm inclusive. The maximum diameter of the shaft within 30 mm from the head is less than half of the diameter of the wide portion of the head, and the length of the shaft is no less than 30 mm. The device can be made of a rigid or semi-rigid material. The device may also include a limiting ball connected to the shaft at no less than 20 mm from the head.

### BRIEF DESCRIPTION OF THE SEVERAL VIEWS OF THE DRAWINGS

FIG. 1 is a side elevational view of a hemorrhoidal treatment device 10 of the present invention.

FIG. 1A is a schematic view illustrating a hemorrhoidal treatment device 10 of the present invention as located within the anal canal.

FIG. 1B is another schematic view illustrating a hemorrhoidal treatment device 10 of the present invention as located within the anal canal.

FIG. 2 is a side elevational view of the hemorrhoidal treatment device 20 of the present invention.

FIG. 2A is a schematic view illustrating a hemorrhoidal treatment device 20 of the present invention as located within the anal canal.

FIG. 2B is another schematic view illustrating a hemorrhoidal treatment device 20 of the present invention as located within the anal canal.

### DETAILED DESCRIPTION OF THE INVENTION

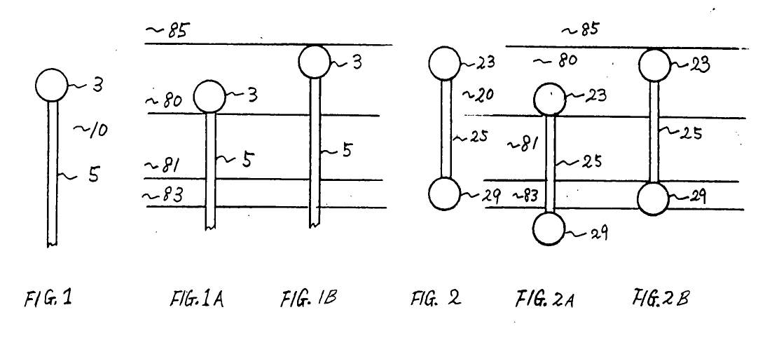

FIG. 1 shows a hemorrhoid treatment device 10 of the present invention. The device 10 is utilized so as to work with the rectal pressure and the pecten within the anal canal. This pecten is a narrow concave section of the anal canal.

The device 10 has a head 3, with a shaft 5 connected to the head 3. The head 3 has a ball-like shape and the diameter of the wide portion of the head 3 is from 6 mm to 14 mm inclusive, while the optimum diameter is 6 mm to 12 mm inclusive. The shaft 5 is generally a cylindrical shape. The diameter of the shaft 5 within 30 mm from the head 3 is less than half of the diameter of the wide portion of the head 3. The optimum diameter of the shaft is less than one third of the diameter of the wide portion of the head. The length of the shaft 5 is between 25 mm and 50 mm, inclusive. The preferred material of the device is a rigid or a semi-rigid material. The semi-rigid materials can be of a soluble material.

The shape of the head can be oval or arrow shape, if such a shape is adopted, then, the head having a wide portion, between the top 2 and the bottom 4 of the head 3 and the length from the top 2 to the bottom 6 of the head 3 is from 6 mm to 16 mm inclusive, while the optimum length is from 6 mm to 12 mm inclusive.

FIGS. 1A and 1B explain the working principle of the device 10 inside the anal canal. The main components of the anal canal are the internal hemorrhoid area 80, the pecten 81 and the subcutaneous external sphincter 83. The pecten 81 is characterized by the formation of a thick hardened ring of fibrous tissue with resulting stenosis and contraction to form a narrow passage wall. The subcutaneous external sphincter 83 contains the external hemorrhoid. Above the internal hemorrhoid area 80 is the ampulla of the rectum 85. The lengths of the main components are: a 12 to 18 mm internal hemorrhoid area; a 12 to 18 mm pecten; and a 5 to 10 mm external sphincter.

FIG. 1A shows when the device 10 is in use. As is inserted into the anal canal, the head 3 reaches the internal hemorrhoid area 80. The head 3 stays at the lower edge of the internal hemorrhoid area 80. This is the first position of the device 10 inside the anal canal. The shaft 5 has a smaller diameter compared to the head 3. This helps the head 3 to be completely wrapped around by the tissues of the internal hemorrhoid area 80. Also, the thrust force of the shaft 5 caused by the lateral pressure of the pecten applied to the shaft 5 has a nominal effect on moving the position of the head 3 because the smaller the size of the shaft it has more surface ratio than the capacity. When the pecten contracts, the pecten tightens around the shaft 5 and moves upward with the shaft 5. When the pecten relaxes, the pecten releases the shaft 5 and the pecten alone moves down to the original position, leaving the shaft in the upward position. The peristaltic movement inside the pecten works better on the small diameter of the shaft and contributes to moving the head 3 up to the upper edge of the internal hemorrhoid area 80. This is the second position of the device 10 inside the anal canal as shown in FIG. 1B. At this time, shaft 5 is through the lower part of the anal canal and the end of the shaft is outside. In this arrangement, the rectal pressure pushes the head 3 down toward the first position as shown in FIG. 1A. The effect of the rectal pressure to the head 3 diminishes as the head 3 moves down toward the pecten. The head 3 is of a relatively small size and has a step at its juncture with the shaft 5, which prevents diminished rectal pressure from pushing the head down through the narrow passage of the pecten 81. These up and down free movements of the head 3 effectively massage the internal hemorrhoid. The ideal diameter of the wide portion of the head 3 is less than two-thirds of the length of the internal hemorrhoid area to ensure optimum movement of the head inside the internal hemorrhoid area 80. If the diameter and the length of the head 3 are larger than the length of the internal hemorrhoid area, the head 3 moves up beyond the upper edge of the internal hemorrhoid area or until the involuntary evacuative rectal pressure pushes out the head 3 from the anal canal.

FIG. 2 shows a first alternative embodiment of the hemorrhoid treatment device 20 of the present invention.

The device 20 has a head 23, with a shaft 25 connected to the head 23. The figures of the head 23 and the shaft 25 are as same as the head 3 and the shaft 5 of the device 10 on the FIG. 1. The device 20 has an auxiliary limiting ball 29 on the end of the shaft 25 opposite the head 23. The limiting ball 29 massages the external hemorrhoids and also ensures a limitation of the free moving distance of the head 23 within the internal hemorrhoid area 80. Since the length of the human anal sphincter is 25 to 40 mm, the distance from the top end 22 of the head 23 to the limiting ball 29 is between 25 mm and 45 mm. The optimum diameter of the wide portion of the limiting ball 29 is no more than two thirds of the diameter of the wide portion of the head 23.

FIG. 2A shows the device 20 inserted into the anal canal as the top ball 23 reaches the internal hemorrhoid area 80, and the limiting ball 29 remains outside the anal canal. This is the first position of the device 20 inside the anal canal. FIG. 2B shows the head 23 moves upwardly by the contraction and the peristaltic movement of the pecten 81 to reach the upper edge of the internal hemorrhoid area 80, and as the head 23 is moving upwardly, the limiting ball 29 moves together upward to massage the external hemorrhoid area 83. This is the second position of the device 20 inside the anal canal. Then the downward rectal pressure pushes the device down to the first position. These up and down movements of the device 20 inside the anal canal massages the internal 80 and the external hemorrhoid gland 83.

1. A hemorrhoid treatment device comprising:
a unitary body having a head connected to one end of a shaft, said head having a spherical shape, said head having a maximum diameter of between 6 mm and 14 mm inclusive, said shaft having a cylindrical shape, said shaft having a diameter that is less than one-half of said maximum diameter of said head, said body having a length from one end to an opposite end of between 25 mm and 45 mm.

2. The device of claim 1, further comprising:
a limiting ball affixed to said apposite end of said body at an end of said shaft.

3. The device of claim 2, said shaft having a constant diameter extending entirely between said head of said limiting ball.

4. The device of claim 2, said limiting ball having a diameter that is no greater than said maximum diameter of said head.

5. The device of claim 4, said limitations ball having a maximum diameter that is less than two thirds of said maximum diameter of said head.

6. The device of claim 2, said limiting ball being integrally formed with said body.

7. The device of claim 1, said unitary body being formed of a polymeric material.

8. A hemorrhoid treatment device comprising:
a body having a head connected to one end of a shaft, said head having a spherical shape, said head having a maximum diameter of between 6 mm and 14 mm inclusive, said shaft having a generally cylindrical shape, said shaft having a maximum diameter that is less than one-half said maximum diameter of said head; and
a limiting ball attached to an opposite end of said shaft, said distance between and end of said head opposite said shaft to said limiting ball being between 25 mm to 45 mm.

9. The device of claim 8, said shaft having a continuously constant diameter extending between said head and said limiting ball.

10. The device of claim 9, said body being integrally formed together of a polymeric material.

11. The device of claim 10, said limiting ball being integrally formed with said body.

12. The device of claim 8, said limiting ball having a maximum diameter that is no greater than said maximum diameter of said head.

13. The device of claim 12, said maximum diameter of said limiting ball being less than two thirds of said maximum diameter of said head.

14. A hemorrhoid treatment device comprising:
a body having a head connected to one end of a shaft, said head having a spherical shape, said head having a maximum diameter of between 6 mm and 14 mm inclusive, said shaft having an entirely cylindrical shape, said shaft having a maximum diameter that is less than one third of said maximum diameter of said head; and
a limiting ball affixed to an opposite end of said shaft, a distance between and end of said head opposite said shaft to said limiting ball being between 25 and 45 mm, said limiting ball having a maximum diameter that is less than two thirds of said maximum diameter of said head.

15. The device of claim 14, said head and said shaft being integrally formed of a polymeric material.

16. The device of claim 14, said limiting ball being integrally formed with said body.

17. The device of claim 14, said shaft having a constant diameter entirely between said head and said limiting ball.
